(19) United States
(12) Patent Application Publication
(10) Pub. No.: US 2016/0000731 A1
(43) Pub. Date: Jan. 7, 2016

(54) SORE-THROAT COMPOSITIONS AND RELATED METHODS

(71) Applicant: Xlear, Inc., Orem, UT (US)

(72) Inventors: Alonzo H. Jones, Plainview, TX (US); Nathan Jones, Orem, UT (US)

(21) Appl. No.: 14/791,106

(22) Filed: Jul. 2, 2015

Related U.S. Application Data
(60) Provisional application No. 62/020,192, filed on Jul. 2, 2014.

(51) Int. Cl. A61K 31/047; A61K 36/484; A61K 36/23; A61K 36/61; A61K 9/00; A61K 47/10

(52) U.S. Cl. CPC A61K 31/047; A61K 9/0053; A61K 47/10; A61K 36/23; A61K 36/61; A61K 36/484

(57) ABSTRACT

Methods for alleviating sore throat conditions and related compositions. In some such compositions, at least one non-hexose sugar alcohol and/or at least one non-hexose sugar alcohol precursor may be present in a concentration effective for reducing a concentration of sore-throat causing microorganisms in a patient's throat. The composition may further comprise at least one coating agent in a concentration effective for prolonging the presence of the at least one non-hexose sugar alcohol and/or at least one non-hexose sugar alcohol precursor in a patient's throat to treat the sore throat condition.

### RELATED APPLICATIONS

This application claims the benefit under 35 U.S.C. §119(e) of U.S. Provisional Patent Application No. 62/020,192 filed Jul. 2, 2014 and titled “SORE-THROAT COMPOSITIONS AND RELATED METHODS,” which application is incorporated herein by reference in its entirety.

### SUMMARY

Disclosed herein are examples of embodiments and implementations of compositions and methods for treating sore throat conditions. In an example of an implementations of a method for treating a sore throat condition, the method may comprise identifying a subject having a sore throat condition and providing a composition for treating the condition, which composition may comprise at least one of a non-hexose sugar alcohol and a non-hexose sugar alcohol precursor, wherein the at least one of a non-hexose sugar alcohol and a non-hexose sugar alcohol precursor is present in a concentration of between about 10% and about 60% by weight. The method may further comprise delivering the composition into the subject's throat to treat the sore throat condition.

In some implementations, the composition may comprise at least one of a non-hexose sugar alcohol and a non-hexose sugar alcohol precursor in a concentration of between about 20% and about 55% by weight. In some such implementations, the composition may comprise at least one of a non-hexose sugar alcohol and a non-hexose sugar alcohol precursor in a concentration of about 50% by weight.

In some implementations, the at least one of a non-hexose sugar alcohol and a non-hexose sugar alcohol precursor may comprise only at least one non-hexose sugar alcohol, such as xylitol and/or erythritol, or just xylitol alone.

In another example of a method for treating a sore throat condition, the method may comprise identifying a subject having a sore throat condition and providing a composition comprising at least one of xylitol, xylose, erythritol, ribose, and arabinose in a concentration of between about 20% and about 55% by weight. The composition may further comprise at least one coating agent, such as at least one of glycerin, sodium alginate, propylene glycol, and carboxymethylcellulose sodium, in a concentration effective for prolonging the presence of the at least one of xylitol, xylose, erythritol, ribose, and arabinose in a patient's throat to treat the sore throat condition. The method may further comprise delivering the composition into the subject's throat to treat the sore throat condition.

In some implementations, the at least one of xylitol, xylose, erythritol, ribose, and arabinose may comprise only xylitol. In some such implementations, the composition may comprise only xylitol in a concentration of about 50% by weight.

In some implementations, the composition may comprise at least one of xylitol, xylose, erythritol, ribose, and arabinose in a concentration of about 50% by weight.

In some implementations, the composition may comprise a viscosity of between about 50 and about 300 centipoise at room temperature.

In an example of a composition for alleviating a sore throat condition according to some embodiments, the composition may comprise at least one non-hexose sugar alcohol and/or at least one non-hexose sugar alcohol precursor in a concentration effective for reducing a concentration of sore-throat causing microorganisms in a patient's throat. In some such embodiments, the composition may solely comprise at least one non-hexose sugar alcohol in a concentration effective for reducing a concentration of sore-throat causing microorganisms in a patient's throat.

Some compositions may further comprise at least one coating agent in a concentration effective for prolonging the presence of the at least one non-hexose sugar alcohol and/or the at least one non-hexose sugar alcohol precursor in a patient's throat to treat the sore throat condition. In some such compositions, the at least one coating agent may comprise at least one of glycerin, sodium alginate, propylene glycol, and carboxymethylcellulose sodium.

Some compositions may further comprise at least one of an expectorant, an anti-mucosal agent, and a mucolytic agent. In some such compositions, the at least one of an expectorant, an anti-mucosal agent, and a mucolytic agent may comprise at least one of eucalyptus oil, licorice root, guaifenesin, guaiacum, phenylephrine HCL, echinacea, and holy basil.

Some compositions may further comprise an antibacterial agent, such as an anise seed extract, a star anise extract, and/or propolis.

Some compositions may comprise a viscosity of between about 30 and about 500 centipoise at room temperature. In some such compositions, the viscosity of between about 50 and about 300 centipoise. In some such compositions, the viscosity of between about 80 and about 200 centipoise.

The features, structures, steps, or characteristics disclosed herein in connection with one embodiment may be combined in any suitable manner in one or more alternative embodiments and/or implementations.

### DETAILED DESCRIPTION

Cough syrups, throat lozenges, throat sprays, and other similar compositions for treating sore throats typically contain sugars, such as sucrose, agave, honey, fructose, glucose, and other hexose sugars. However, it has been discovered that such sugars may serve as an energy source for certain species of bacteria, which may contribute to, rather than alleviate, certain sore throat conditions.

However, certain non-hexose sugar alcohols, such as xylitol and erythritol, for example, along with various non-hexose sugar alcohol precursors, such as xylose, ribose, and arabinose, for example, have been discovered to have a sweetness equivalent to that of sucrose, but to also possess unique properties that render it unsuitable as a source of energy for certain bacteria and/or other microorganisms. More particularly, without being limited by theory, it is thought that, due to its five-carbon sugar alcohol structure, xylitol, for example, cannot be consumed or otherwise be used as an energy source for most oral microorganisms. Regular consumption of xylitol has also been shown to reduce the incidence of dental caries. This is primarily attributed to xylitol's ability to inhibit and/or reduce the growth and acid production of S. mutans, which is thought to be one of the more important bacterium taking part in the pathogenesis of dental caries.

Xylitol has also been demonstrated to inhibit the growth of Streptococcus pneumonia in vitro during its logarithmic growth phase. The Streptococcus pneumonia bacteria species is believed to be the causative agent of certain types of pneumonia and upper respiratory infections, and is also associated with other infectious diseases, such as meningitis and sepsis.

Although certain other sweeteners, such as saccharine, may not be consumed by certain bacteria and/or other oral microorganisms, one or more of the sweeteners disclosed herein may be more useful in actively, rather than passively, starving these oral microorganisms than saccharine and other such sweeteners that are typically not consumed by certain oral microorganisms. This is because the inventors have discovered that xylitol, erythritol, and other such agents disclosed herein may be consumed by certain oral microorganisms known to cause sore throats and/or associated infectious diseases but cannot be broken down by such microorganisms so as to be usable as a source of energy. In other words, without being limited by theory, it is thought that the sweeteners disclosed herein may be useful in treating sore throat conditions and/or other oral infections and/or problems by causing certain microorganisms to ingest or consume the sweetener in place of other food/energy sources. This may result in faster eradication of such microorganisms that often result in sore throat symptoms. In some embodiments and implementations, by using such sweeteners in combination with other active ingredients for treating the symptoms and/or cause of sore throats, an even more improved composition may be created.

It is therefore thought that use of xylitol or another non-hexose sugar alcohol, such as xylitol, erythritol, or a non-hexose sugar alcohol precursor, such as xylose, ribose, or arabinose, in conjunction with other active ingredients known to relieve symptoms of sore throats, may result in an improved composition for use in treating such symptoms and/or treating the cause of such symptoms. More particularly, by using a sweetener that enhances the ability of other agents to treat sore throats by actively starving the microorganisms causing the symptoms, rather than one that counteracts the active ingredients in a sore throat composition by feeding the microorganisms, or one that only passively starves the microorganisms by providing a sweetener that is not consumed by the more common oral and pharyngeal pathogens, compositions using the principles disclosed herein may result in improved ability to treat sore throat symptoms and, more importantly, to fight the bacterial and other microorganisms behind these symptoms.

Consistent with embodiments disclosed herein, certain non-hexose sugar alcohols, such as xylitol and/or erythritol, and/or a non-hexose sugar alcohol precursor, such as xylose, ribose, or arabinose, may be administered to treat symptoms of a sore throat, preferably in conjunction with other sore throat treatment active ingredients and/or compositions to provide a synergistic treatment effect, rather than a detracted treatment effect, as current sore throat formulations provide. These compositions may not only enhance treatment and provide this synergistic treatment effect, but may also provide an improved, or at least similar, taste by virtue of including the disclosed sweetener agents.

Some preferred embodiments and implementations may comprise a combination of pentose sugar alcohols. Xylitol and/or erythritol alone may be included in some such preferred embodiments and implementations. Xylitol alone may be included in some such preferred embodiments and implementations.

In some embodiments and implementations, sore-throat treatment compositions disclosed herein may comprise methods, agents, compositions, etc. disclosed in U.S. Pat. Nos. 6,054,143 and 6,258,372, both titled “XYLITOL NOSE SPRAY” and U.S. Pat. No. 6,599,883 titled “NASAL DELIVERY OF XYLITOL,” each of which is incorporated herein by reference in its entirety.

Some of the sweeteners disclosed herein, such as xylitol in particular, have also been demonstrated to be very effective in moisturizing mucous-lined passages and cavities, such as the oral cavity and throat passage. Without being limited by theory, this is thought to occur because xylitol can create a hyper-osmotic solution that pulls moisture towards it from surrounding tissues without generated mucous. Thus, some embodiments and implementations disclosed herein may also result in improved ability to add moisture and/or prevent unwanted dryness, along with the accompanying bacterial starvation benefits discussed above.

In some preferred embodiments and implementations, a threshold concentration of one or more non-hexose sugar alcohols, such as xylitol and/or erythritol, and/or a non-hexose sugar alcohol precursor, such as xylose, ribose, or arabinose, may be used. For example, in some embodiments and implementations, the composition may comprise at least about 5% by weight of a non-hexose sugar alcohol and/or a non-hexose sugar alcohol precursor. In some such embodiments and implementations, the composition may comprise at least about 5% by weight of xylitol, erythritol, xylose, ribose, and/or arabinose. In some such embodiments and implementations, the composition may comprise at least about 5% by weight of a pentose sugar alcohol. In some such embodiments and implementations, the composition may comprise at least about 5% by weight of xylitol and/or erythritol. In some such embodiments and implementations, the composition may comprise at least about 5% by weight of xylitol.

In some embodiments and implementations, the composition may comprise at least about 10% by weight of a non-hexose sugar alcohol and/or a non-hexose sugar alcohol precursor. In some such embodiments and implementations, the composition may comprise at least about 10% by weight of xylitol, erythritol, xylose, ribose, and/or arabinose. In some such embodiments and implementations, the composition may comprise at least about 10% by weight of a pentose sugar alcohol. In some such embodiments and implementations, the composition may comprise at least about 10% by weight of xylitol and/or erythritol. In some such embodiments and implementations, the composition may comprise at least about 10% by weight of xylitol.

In some embodiments and implementations, the composition may comprise between about 10% and about 60% by weight of a non-hexose sugar alcohol and/or a non-hexose sugar alcohol precursor. In some such embodiments and implementations, the composition may comprise between about 10% and about 60% by weight of xylitol, erythritol, xylose, ribose, and/or arabinose. In some such embodiments and implementations, the composition may comprise between about 10% and about 60% by weight of a pentose sugar alcohol. In some such embodiments and implementations, the composition may comprise between about 10% and about 60% by weight of xylitol and/or erythritol. In some such embodiments and implementations, the composition may comprise between about 10% and about 60% by weight of xylitol.

In some embodiments and implementations, the composition may comprise between about 20% and about 55% by weight of non-hexose sugar alcohol and/or a non-hexose sugar alcohol precursor. In some such embodiments and implementations, the composition may comprise between about 20% and about 55% by weight of xylitol, erythritol, xylose, ribose, and/or arabinose. In some such embodiments and implementations, the composition may comprise between about 20% and about 55% by weight of a pentose sugar alcohol. In some such embodiments and implementations, the composition may comprise between about 20% and about 55% by weight of xylitol and/or erythritol. In some such embodiments and implementations, the composition may comprise between about 20% and about 55% by weight of xylitol.

In some embodiments and implementations, the composition may comprise about 50% by weight of non-hexose sugar alcohol and/or a non-hexose sugar alcohol precursor. In some such embodiments and implementations, the composition may comprise about 50% by weight of xylitol, erythritol, xylose, ribose, and/or arabinose. In some such embodiments and implementations, the composition may comprise about 50% by weight of a pentose sugar alcohol. In some such embodiments and implementations, the composition may comprise about 50% by weight of xylitol and/or erythritol. In some such embodiments and implementations, the composition may comprise about 50% by weight of xylitol.

In some preferred embodiments and implementations, the composition may be presented and ingested in the form of a syrup or other relatively thick liquid. Although some embodiments and implementations are contemplated in which the composition may comprise other delivery mechanisms, such as a spray, it is thought that the benefits described above relating to active starvation of sore-throat causing microorganisms may be most effective when used in a liquid form that may be used to coat the throat and/or other linings of the oral cavity. By providing a thick, viscous liquid, such as a syrup, the active starvation benefits described herein may be enhanced, since the xylitol or other similar sweetener that is ingested but not digested by certain sore-throat causing microorganisms may then linger within the throat, thereby continuing to actively starve these microorganisms. In compositions comprising other active, sore-throat treatment agents, this may result in a synergistic effect that may lead to an increased ability to fight sore throat symptoms and/or infections relative to those agents used alone, or in combination with other sweeteners that are either ingested and digested, or are not ingested at all, by certain microorganisms.

Because the viscosity may be important for certain applications and embodiments, in certain preferred embodiments and implementations, the viscosity of the composition may be between about 30 and about 500 centipoise at room temperature. In some such embodiments and implementations, the viscosity of the composition may be between about 50 and about 300 centipoise at room temperature. In some such embodiments and implementations, the viscosity of the composition may be between about 80 and about 200 centipoise at room temperature.

In certain embodiments and implementations, a solution comprising one or more non-hexose sugar alcohols, such as xylitol, xylose, erythritol, ribose, and/or arabinose, and one or more sore-throat treatment agents may further comprise a buffer, a thickening agent, a bio-adhesive, and/or a humectant. A pharmaceutically acceptable surfactant and a preservative may also be included along with one or more excipients suitable for a pharmaceutical composition.

In embodiments including a buffer, the buffer may be configured to maintain a pH level of the solution. Exemplary suitable buffers include acetate, citrate, and phosphate buffers. The thickening agent may include, for example, one or more of methylcellulose, xanthan gum, carboxyl methylcellulose, polyvinyl alcohol, hydroxpropyl cellulose, carbomer, starches, chitosans, acrylates, and mixtures thereof. In certain embodiments, these substances may also act as suitable bio-adhesives. Suitable exemplary humectants include sorbitol, propylene glycol, glycerol, and/or any combination thereof. Suitable surfactants may be anionic, cationic, or nonionic, and may include polyoxyethylene derivatives, fatty acids, and/or partial esters of sorbitol anhydrides. For example, the surfactant may include sodium lauryl sulfate, polysorbate 80, polyoxyl stearate, polyoxy ethylene 50, fusieates, bile salts, and octoxynol. However, it should also be understood that many embodiments of the compositions disclosed herein will not need to include a buffer.

In certain embodiments and implementations, the composition may further comprise one or more ingredients configured for use in coating the throat and/or other parts of the oral cavity. For example, some embodiments may comprise glycerin, sodium alginate, propylene glycol, and/or carboxymethylcellulose sodium. These ingredients facilitate keeping the xylitol, erythritol, or other similar ingredient described herein, in the throat/oral cavity to prolong its efficacy.

Some embodiments and implementations may further comprise one or more expectorants, anti-mucosal agents, or mucolytic agents, such as eucalyptus oil, licorice root, guaifenesin, guaiacum, phenylephrine HCL, echinacea, and/or holy basil.

Some embodiments and implementations may further comprise one or more antibacterial agents, such as anise oil or other anise seed extracts, star anise (illicium verum) extracts, and/or propolis.

Some embodiments and implementations may further comprise one or more anti-inflammatory agents, such as marshmallow root, acetametaphine, ibuprofen, and/or aspirin.

Some embodiments and implementations may further comprise one or more cough suppressants, such as thyme, dextromethorphan, codeine, cuprum sulphuricum, drosera rotundifolia, and/or ipecacuanha.

Some embodiments and implementations may further comprise one or more anti-foaming agents, such as simethicone.

Some embodiments and implementations may further comprise one or more other ingredients used to treat symptoms of the common cold and/or flu, such as echinacea, yerba santa, andrographis paniculata, elderberry (sambucus nigra), ginseng, and/or eleuthero.

Some embodiments and implementations may further comprise one or more analgesics, such as acetaminophen, NSAIDs, lidocaine, other analgesics, and the like.

Some embodiments and implementations may further comprise psyllium seed.

In most preferred compositions, the non-hexose sugar alcohol may comprise xylitol, erythritol, or a combination of xylitol and erythritol.

### EXAMPLE 1

In a more particular example of a composition for treating sore throats, the composition may comprise the following ingredients in at least approximately the concentrations (by weight) presented in the chart below.

| | |
|---|---|
| Xylitol | 50% |
| Water | 20.1% |
| Glycerin | 20% |
| Marshmallow powdered extract | 4% |
| Sodium alginate | 2% |
| Echinacea powdered extract | 1% |
| Anise oil | 0.6% |
| Eucalyptus oil | 0.6% |
| Potassium sorbate | 0.6% |
| Soybean lecithin | 0.5% |
| Flavor | 0.5% |
| Simethicone | 0.1% |

### EXAMPLE 2

In another more particular example of a composition for treating sore throats, the composition may comprise the following ingredients in at least approximately the concentrations (by weight) presented in the chart below.

| | |
|---|---|
| Xylitol | 50% |
| Water | 20.1% |
| Glycerin | 20% |
| Thyme aqueous extract | 4% |
| Sodium alginate | 2% |
| Licorice powdered extract | 1% |
| Anise oil | 0.6% |
| Eucalyptus oil | 0.6% |
| Potassium sorbate | 0.6% |
| Soybean lecithin | 0.5% |
| Flavor | 0.5% |
| Simethicone | 0.1% |

### EXAMPLE 3

In still another more particular example of a composition for treating sore throats, the composition may comprise the following ingredients in at least approximately the concentrations (by weight) presented in the chart below.

| | |
|---|---|
| Xylitol | 50% |
| Glycerin | 20% |
| Anise oil | 0.6% |
| Eucalyptus oil | 0.6% |
| Licorice powdered extract | 1% |
| Marshmallow powdered extract | 4% |
| Potassium sorbate | 0.6% |
| Soybean lecithin | 0.5% |
| Flavor 1 | 0.5% |
| Simethicone | 0.1% |
| Sodium alginate | 2% |
| Water | 19.6% |
| Flavor 2 | 0.5% |

It will be understood by those having skill in the art that changes may be made to the details of the above-described embodiments without departing from the underlying principles presented herein. For example, the compositions disclosed herein may be administered via liquid drops from a dropper, topically (in some cases using a cotton swab or the like), orally, via a mister or atomizer, and/or via any other suitable manner of administration. In addition, any suitable combination of various embodiments, or the features thereof, is contemplated.

Any methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein. The scope of the present invention should, therefore, be determined only by the following claims.

1. A method for treating a sore throat condition, the method comprising the steps of:
identifying a subject having a sore throat condition;
providing a composition comprising at least one of a non-hexose sugar alcohol and a non-hexose sugar alcohol precursor, wherein the at least one of a non-hexose sugar alcohol and a non-hexose sugar alcohol precursor is present in a concentration of between about 10% and about 60% by weight; and
delivering the composition into the subject's throat to treat the sore throat condition.

2. The method of claim 1, wherein the composition comprises at least one of a non-hexose sugar alcohol and a non-hexose sugar alcohol precursor in a concentration of between about 20% and about 55% by weight.

3. The method of claim 2, wherein the composition comprises at least one of a non-hexose sugar alcohol and a non-hexose sugar alcohol precursor in a concentration of about 50% by weight.

4. The method of claim 1, wherein the at least one of a non-hexose sugar alcohol and a non-hexose sugar alcohol precursor comprises only at least one non-hexose sugar alcohol.

5. The method of claim 4, wherein the at least one non-hexose sugar alcohol comprises only at least one of xylitol and erythritol.

6. The method of claim 5, wherein the at least one non-hexose sugar alcohol comprises only xylitol.

7. A method for treating a sore throat condition, the method comprising the steps of:
identifying a subject having a sore throat condition;
providing a composition comprising:
at least one of xylitol, xylose, erythritol, ribose, and arabinose in a concentration of between about 20% and about 55% by weight; and
at least one coating agent comprising at least one of glycerin, sodium alginate, propylene glycol, and carboxymethylcellulose sodium in a concentration effective for prolonging the presence of the at least one of xylitol, xylose, erythritol, ribose, and arabinose in a patient's throat to treat the sore throat condition; and
delivering the composition into the subject's throat to treat the sore throat condition.

8. The method of claim 7, wherein the at least one of xylitol, xylose, erythritol, ribose, and arabinose comprises only xylitol.

9. The method of claim 8, wherein the composition comprises xylitol in a concentration of about 50% by weight.

10. The method of claim 7, wherein the composition comprises at least one of xylitol, xylose, erythritol, ribose, and arabinose in a concentration of about 50% by weight.

11. The method of claim 7, wherein the composition comprises a viscosity of between about 50 and about 300 centipoise at room temperature.

12. A composition for alleviating a sore throat condition, the composition comprising:
at least one non-hexose sugar alcohol in a concentration effective for reducing a concentration of sore-throat causing microorganisms in a patient's throat; and
at least one coating agent in a concentration effective for prolonging the presence of the at least one non-hexose sugar alcohol in a patient's throat to treat the sore throat condition.

13. The composition of claim 12, wherein the at least one coating agent comprises at least one of glycerin, sodium alginate, propylene glycol, and carboxymethylcellulose sodium.

14. The composition of claim 12, further comprising at least one of an expectorant, an anti-mucosal agent, and a mucolytic agent.

15. The composition of claim 14, wherein the at least one of an expectorant, an anti-mucosal agent, and a mucolytic agent comprises at least one of eucalyptus oil, licorice root, guaifenesin, guaiacum, phenylephrine HCL, echinacea, and holy basil.

16. The composition of claim 12, further comprising an antibacterial agent.

17. The composition of claim 16, wherein the antibacterial agent comprises at least one of an anise seed extract, a star anise extract, and propolis.

18. The composition of claim 17, wherein the antibacterial agent comprises anise oil.

19. The composition of claim 12, wherein the composition comprises a viscosity of between about 50 and about 300 centipoise at room temperature.

20. The composition of claim 19, wherein the composition comprises a viscosity of between about 80 and about 200 centipoise at room temperature.
